(19) United States
(12) Patent Application Publication
(10) Pub. No.: US 2009/0130679 A1
(43) Pub. Date: May 21, 2009

(54) Automated system and method for processing genetic material

(75) Inventors: Yu-Min Wu, Taipei County (TW); Yi-Chin Tsai, Banciao City (TW); Hsiao-Cheng Lin, Hsinchu City (TW)

Correspondence Address: REED SMITH LLP, Suite 1400, 3110 Fairview Park Drive, Falls Church, VA, 22042, US

(73) Assignee: Quanta Computer Inc.

(21) Appl. No.: 12/155,789

(22) Filed: Jun. 10, 2008

(30) Foreign Application Priority Data
Nov. 20, 2007 (TW) 096143810

(51) Int. Cl. C12Q 1/68; C12M 1/00

(52) U.S. Cl. 435/6; 435/287.2

(57) ABSTRACT

The invention discloses an automated system and method for processing genetic material. Additionally, the automated system and method of the invention can extract a target genetic material from a sample; amplifying a target nucleic acid sequence from the genetic material; detecting the target nucleic acid by an optical detection module to qualify and/or quantify the target nucleic acid immediately.

### BACKGROUND OF THE INVENTION

1. Field of the Invention

The present invention relates generally to an automated system and method for processing genetic materials, and more particularly, to a system and method for automatically extracting and amplifying genetic materials.

2. Description of the Prior Art

Genome sequencing and decoding of several organisms have recently been performed in many research institutes. With the understanding of the sequence, function, structure, and other characteristics of genetic material, such as nucleic acids (e.g. deoxyribonucleic acid (DNA) or ribonucleic acid (RNA)), the detection methods based on said genetic material are widely developed. Generally speaking, said detection methods are used to detect specific nucleic acid sequences or amino acid sequences in samples such as blood, tissue fluid, and cell culture solution.

In the prior art, the detection method comprises several steps to detect the presence of a specific nucleic acid sequence in a sample, or to quantify the specific nucleic acid sequence in the sample. Particularly, the method can further be used to speculate the presence of specific organisms, such as bacteria, fungus, virus, and a number of said specific organisms.

Before the detection, we have to extract the nucleic acid sequence from the sample and perform polymerase chain reaction (PCR) with specific probes to amplify the target nucleic acid sequence.

However, the complex processes of the above-mentioned extraction and analysis will increase the possibility of artificial error and bias. Moreover, the time needed by the processes could be very long and the accuracy of analysis would be affected.

### SUMMARY OF THE INVENTION

Accordingly, a scope of the present invention is to provide an automated system and method for processing genetic material to solve the problems of the prior art.

According to a preferred embodiment, the automated system for processing genetic material of the invention includes a platform, a pipetting module, an extracting module, a temperature-control module, an optical detection module, and a processing module.

Furthermore, the platform includes a first set of receptacles, each of which is capable of containing a sample, and the pipetting module comprises at least a pipet for sucking and moving at least a first reagent to the first set of receptacles. Additionally, the extracting module is applied to cooperate with the first reagent to extract a target genetic material from the sample. The temperature-control module is used for providing a plurality of thermal cycles and cooperating with at least a second reagent to amplify a target nucleic acid sequence of the target genetic material. The optical detection module is applied for detecting the presence of the target nucleic acid sequence, and generating a detecting signal. The processing module can receive the detecting signal, and it can also qualify and/or quantify the target nucleic acid sequence. Particularly, in an embodiment, the optical detection module can detect the presence, the quantity, or the quality of the target nucleic acid sequence during amplification.

According to another preferred embodiment, the automated method for processing genetic material comprises the automated steps of: (a) mixing a sample with at least a first reagent to extract a target genetic material from the sample; (b) mixing the target genetic material with at least a second reagent; (c) providing a plurality of thermal cycles to amplify a target nucleic acid sequence of the target genetic material; (d) detecting the presence of the target nucleic acid sequence and generating a detecting signal; and (e) receiving the detecting signal and qualifying and/or quantifying the target nucleic acid sequence. Particularly, in an embodiment, step (c) to step (e) can be optionally performed either simultaneously or sequentially.

The objective of the present invention will no doubt become obvious to those of ordinary skill in the art after reading the following detailed description of the preferred embodiment, which is illustrated in the various figures and drawings.

### BRIEF DESCRIPTION OF THE APPENDED DRAWINGS

FIG. 1 illustrates the automated system for processing genetic material of an embodiment of the invention.

FIG. 2 illustrates the side view of the automated system for processing genetic material in FIG. 1.

FIG. 3 illustrates the front view of the automated system for processing genetic material in FIG. 1.

FIG. 4A to 4F illustrate the automated method for processing genetic material of an embodiment of the invention.

### DETAILED DESCRIPTION OF THE INVENTION

The invention provides an automated system and method for processing genetic material. Reference will now be made in detail to the present preferred embodiments of the invention, examples of which are illustrated in the accompanying drawings. Wherever possible, the same reference numbers are used in the drawings and the description to refer to the same or like parts.

According to an embodiment of the invention, the automated system for processing genetic material can include a platform, a pipetting module, an extracting module, a temperature-control module, an optical detection module, and a processing module.

The platform includes a first set of receptacles, and each of the receptacles can contain a sample. In practice, the sample can be any suitable liquid, solid or living sample. The liquid sample can be, but not limited to, blood, saliva, tissue fluid, cell culture solution, microorganism culture solution, and other suitable liquid samples; the solid sample can be, but not limited to, tissue slide, organ slide, and other suitable solid samples; and the living sample can be, but not limited to, nematode (e.g. Caenorhabditis elegans), fish eggs, embryo, and other suitable living samples.

The pipetting module includes at least a pipet, for sucking and moving at least a first reagent to the first set of receptacles. In practice, the pipetting module can be performed in the form of robotic pipettor. Moreover, in practice, the pipetting module includes a plurality of pipets with different scales.

The extracting module can cooperate with the first reagent to extract a target genetic material from the sample. Practically, the first reagent can be, but not limited to, PK solution, cell lysis buffer, wash buffer, elution buffer, magnetic particle solution, and other suitable reagents. Particularly, in practice, the above-mentioned magnetic particle solution contains many magnetic particles, and the surface of the magnetic particles can be coated with nucleic acid absorbing material (e.g. silicon) to absorb nucleic acids. Furthermore, the pH value and the ion concentration of the magnetic particle solution can be suitably adjusted to regulate the absorbing strength between the magnetic particles and the nucleic acids.

The temperature-control module can provide settings of different temperature to the above-mentioned platform and/or the extracting module to assist the extraction. Furthermore, the temperature-control module can further provide a plurality of thermal cycles and cooperates with at least a second reagent to amplify a target nucleic acid sequence of the target genetic material.

The optical detection module can detect the presence of the above-mentioned target nucleic acid sequence, and further detects the quality or quantity of the target nucleic acid sequence. Particularly, the optical detection module can simultaneously perform the detection when the temperature-control module provides the thermal cycles, or after the temperature-control module provides the thermal cycles.

The processing module, such as a micro-control unit (MCU) or central control unit (CPU), is connected to the optical detection module, for receiving the detecting signal, and qualifying and/or quantifying the target nucleic acid sequence in accordance with the detecting signal.

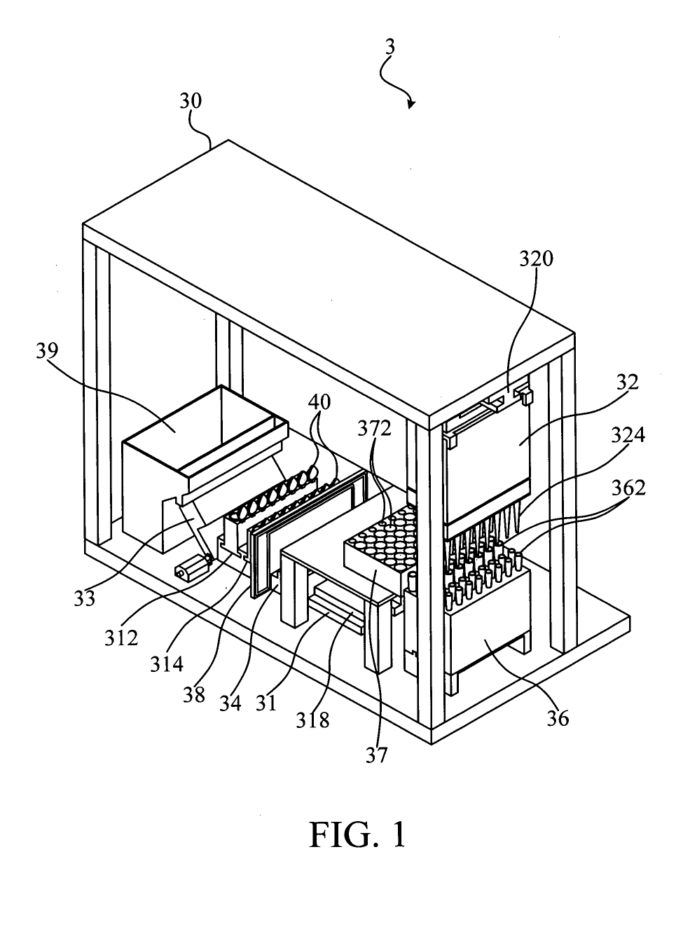

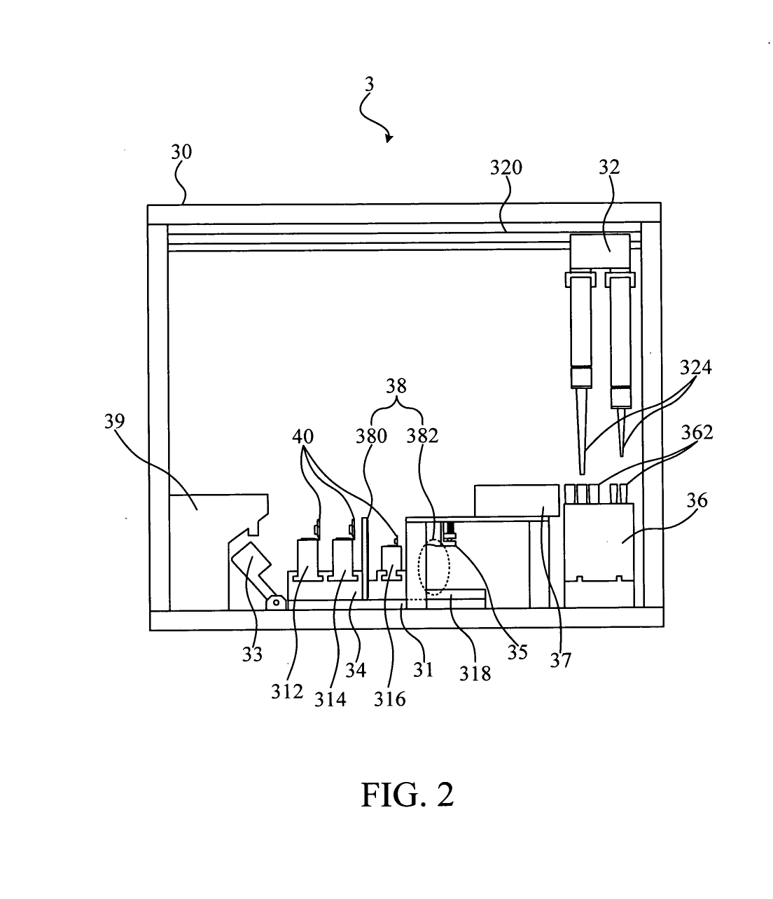

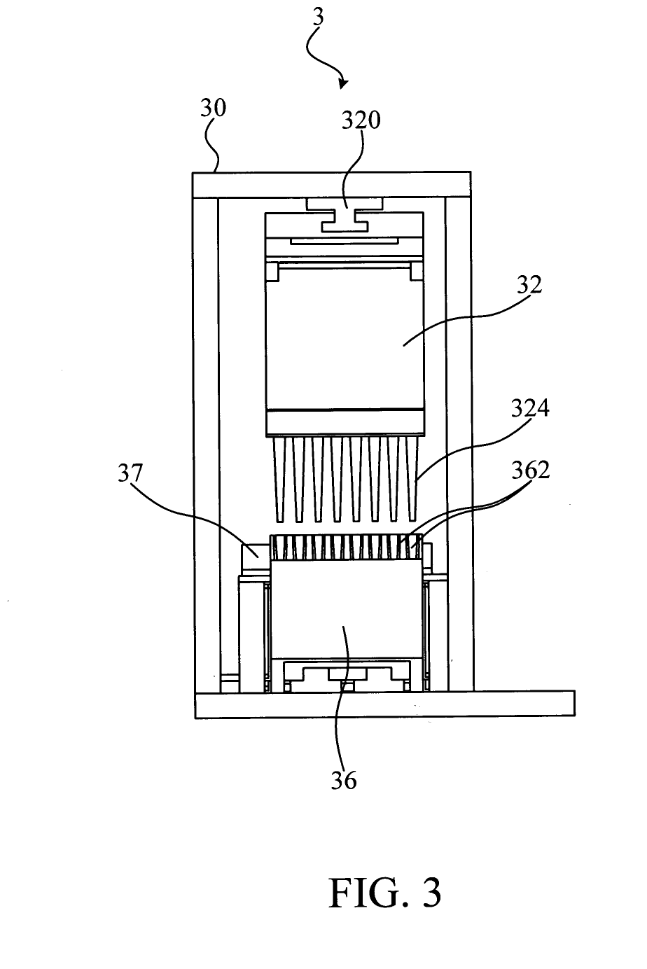

Please refer to FIG. 1 to FIG. 3. FIG. 1 illustrates an automated system for processing genetic material of an embodiment of the invention; FIG. 2 illustrates the side view of the automated system in FIG. 1; and FIG. 3 illustrates the front view of the automated system in FIG. 1. In the embodiment, the automated system 3 for the processing of genetic material includes a housing 30, a platform 31, a pipetting module 32, an extracting module 33, a temperature-control module 34, an optical detection module 35, a pipet tip container 36, a reagent container 37, a PCR reaction module 38, and a waste-discarding region 39.

In the embodiment, a first set of receptacles 312, a second set of receptacles 314, and a third set of receptacles 316 are disposed on the platform 31. In practice, the first set of receptacles 312 can be formed of a non-magnetic material with high thermal conductivity (such as, but not limited to, aluminum and silver). The first set of receptacles 312 can contain a plurality of eppendorf tubes 40, and each of the eppendorf tubes 40 can contain a sample. In practice, the second set of receptacles 314 can be formed of a non-magnetic material with low thermal conductivity (such as, but not limited to, acrylic material). The second set of receptacles 314 can also contain a plurality of eppendorf tubes 40, and each of the eppendorf tubes 40 can contain the target genetic material extracted from the sample. In practice, the third set of receptacles 316 can be formed of a non-magnetic material with high thermal conductivity (such as, but not limited to, aluminum and silver). The third set of receptacles 316 can also contain a plurality of eppendorf tubes 40, and each of the eppendorf tubes 40 can contain the mixture of the target genetic material and second reagent.

The pipet tip container 36 contains a plurality of tips 362 which each has a scale (e.g. 10 μl, 20 μl, 50 μl, 100 μl, 200 μl, 500 μl, 1000 μl, or optionally other scales). The reagent container 37 comprises a plurality of reagent receptacles 372 for containing the first reagent and/or the second reagent.

Additionally, the pipetting module 32 includes several pipets 324 with different ranges of scale. Moreover, the pipetting module 32 can be connected to the moving module 320, so that the moving module 320 can horizontally or vertically move the pipetting module and positioning the pipetting module 32. Accordingly, the pipetting module 32 can selectively use one of the pipets 324 to wedge suitable tip 362 on the pipet tip container 36 to suck and/or move the sample and the reagent in the reagent receptacles 372.

Particularly, all of the above-mentioned first set of receptacles 312, second set of receptacles 314, and third set of receptacles 316 can be designed to be removable. Furthermore, part of the first reagents can be added in the eppendorf tubes 40 of the first set of receptacles 312 in advance, and part or all of the second reagents can be added in the eppendorf tubes 40 of the third set of receptacles 316 in advance, so as to reduce the moving frequency of the pipetting module 32 and the operation time of the system of the invention.

In the embodiment, the temperature-control module 34 is disposed between the platform 31 and the sets of receptacles 312, 314 and 316, to provide suitable temperature to the sets of receptacles 312, 314 and 316. Furthermore, the moving module 318 can be connected to the platform 31, so as to horizontally or vertically move the platform 31 and position the platform 31.

As shown in FIG. 1 and FIG. 2, the extracting module 33 in the embodiment is rotatably mounted adjacent to the platform 31, so that the extracting module 33 is optionally disposed close to the first set of receptacles 312. Particularly, when the first reagent includes a magnetic particle solution, the extracting module 33 (e.g. comprises strong permanent magnet) can generate a magnetic field adjacent to the first set of receptacles 312 after the mixture of the sample and the first reagent in the first set of receptacles 312. The magnetic field can fix the magnetic particles with the target nucleic acid on the inner wall of the first set of receptacles 312. At that time, the pipetting module 32 can remove residues other than the magnetic particles to reach the goal of isolating the target nucleic acid from the sample.

The PCR reaction module 38 includes a reaction chamber 382 and a door 380 of the reaction chamber 382. The reaction chamber 382 can contain the third set of receptacles 316 which comprises eppendorf tubes 40 containing the second reagent and the target genetic material. The door 380 can be disposed closest to the opening of the reaction chamber 382, so as to seal the second reagent and the target genetic material in the reaction chamber 382. Moreover, the PCR reaction module 38 receives the thermal cycles provided by the temperature-control module 34 to amplify the target nucleic acid sequence.

When the above-mentioned PCR reaction module 38 performs the amplification process, the optical detection module 35 can perform optical detection of the eppendorf tubes 40 on the third set of receptacles 316, so as to immediately detect the presence of the target nucleic acid sequence. As mentioned above, the optical detection module 35 can perform optical detection after the amplification processes. Particularly, in practice, the optical detection module 35 has a special structure to assist the PCR reaction module 38 to seal the mixture of the second reagent and the target genetic material in the reaction chamber 382. Moreover, the heating mechanism of the optical detection module 35 can prevent the steam from congealing on the inner wall of the eppendorf tubes 40 of the third set of receptacles 316 or evaporating. Furthermore, the waste-discarding region 39 can contain the wasted material or liquid discarded by the pipetting module 32.

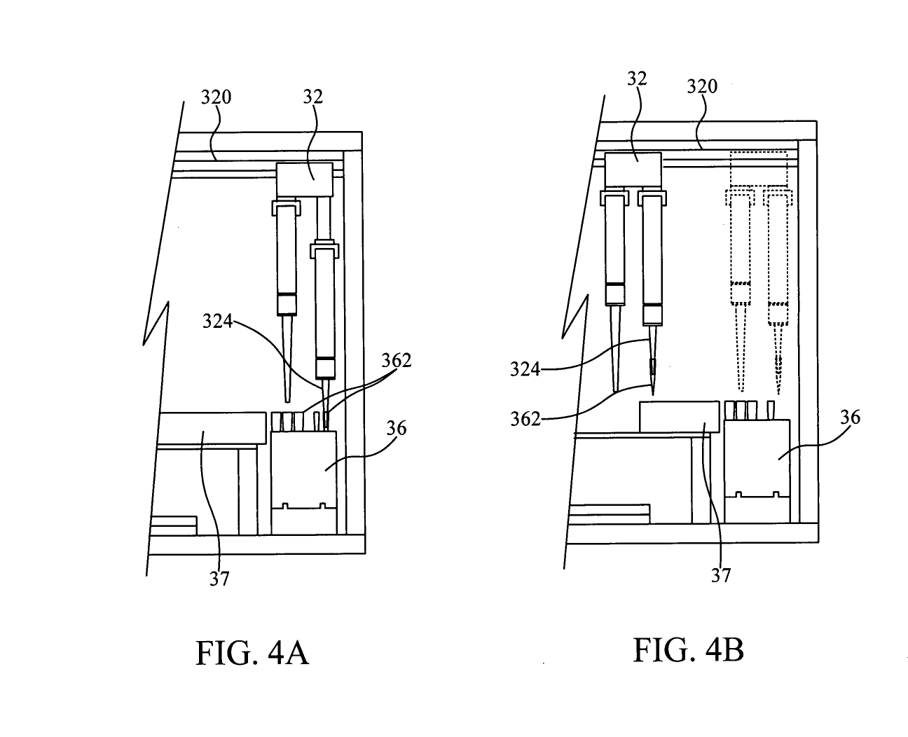

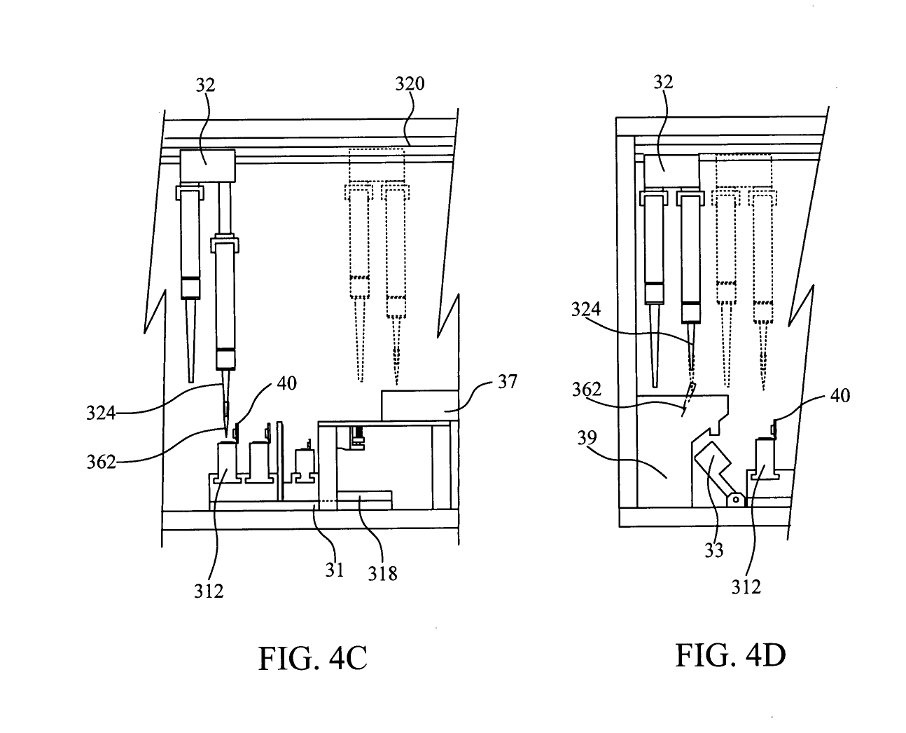

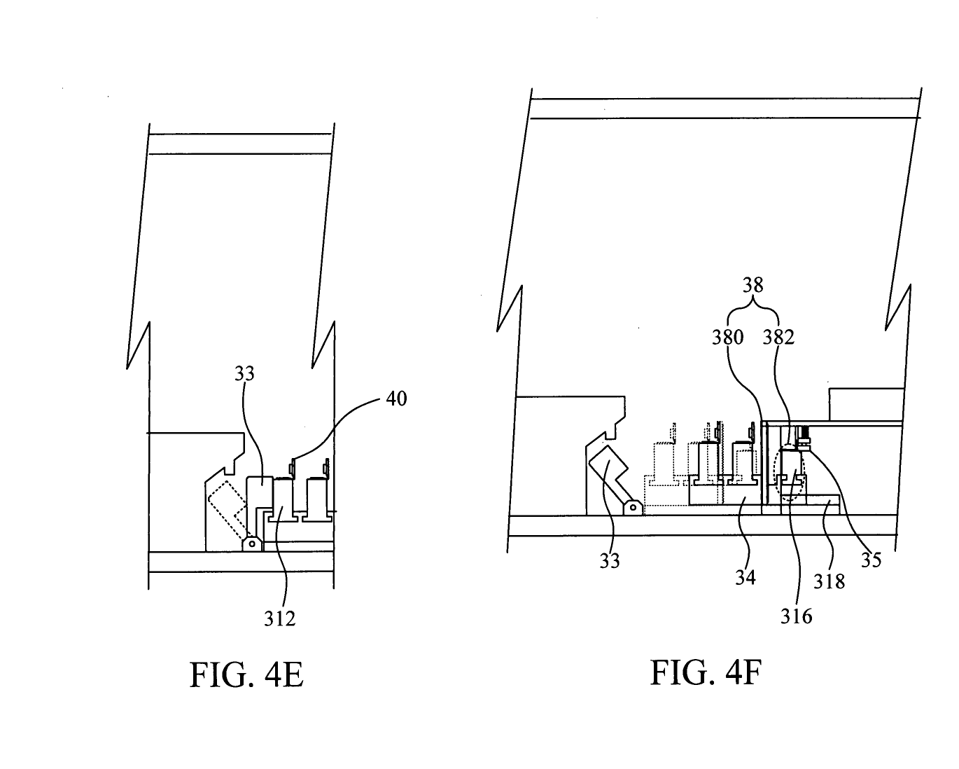

Please refer to FIG. 4A to 4F which illustrate the automated steps for processing genetic material of an embodiment of the invention. In the embodiment, the sample has been added in the eppendorf tubes 40 of the first set of receptacles 312.

First of all, as shown in FIG. 4A, the moving module 320 horizontally moves the pipetting module 32 to the top of the pipet tip container 36, and vertically moves the pipet 324 with suitable scale of the pipetting module 32 to wedge the tip 362 matching with the pipet 324. Afterward, as shown in FIG. 4B, the moving module 320 horizontally moves the pipetting module 32 to the top of the reagent container 37, and then vertically moves the above-mentioned pipet 324 with the tip 362 to let the pipetting module 32 suck suitable volume of PK solution.

Consequently, as shown in FIG. 4C, the moving module 320 horizontally moves the pipetting module 32 to the top of the eppendorf tubes 40 of the first set of receptacles 312, and then vertically moves the above-mentioned pipet 324, so that the tip 362 wedged thereon can be moved close to the eppendorf tubes 40. At that time, the pipetting module 32 adds the PK solution to the eppendorf tubes 40, and mixes the PK solution and the sample. Furthermore, as shown in FIG. 4D, the moving module 320 horizontally moves the pipetting module 32 to the top of the waste-discarding region 39, and the pipetting module 32 discards the used tip 362 to the waste-discarding region 39.

By the similar processes as mentioned above, the pipetting module 32 further adds suitable volume of cell lysis buffer in the eppendorf tubes 40 of the first set of receptacles 312 and mixes the cell lysis buffer with the PK solution and the sample. Furthermore, the temperature-control module 34 can optionally heat the first set of receptacles 312 to assist the cell in the sample to be lysed. The pipetting module 32 further adds suitable volume of magnetic particle solution in the eppendorf tubes 40 of the first set of receptacles 312, so that the target genetic material can be adhered on the surface of magnetic particles. Afterward, as shown in FIG. 4E, the extracting module 33 rotates to be close to the first set of receptacles 312, and enforces a magnetic field to the first set of receptacles 312, so that the magnetic particles with the target genetic material attached on the inner wall of the eppendorf tubes 40. Afterward, the extracting module 33 maintains the position in FIG. 4E, and the pipetting module 32 removes residues other than the magnetic particles to the waste-discarding region 39.

Consequently, the extracting module 33 rotates to the position as shown in FIG. 4D, and the pipetting module 32 adds wash buffer in the above-mentioned eppendorf tubes 40 to wash the magnetic particles. Afterward, the extracting module 33 rotates to be moved close to the first set of receptacles 312 and enforces magnetic field again, and allows the magnetic particles with the target genetic material be attached on the inner wall of the eppendorf tubes 40. Again, the pipetting module 32 removes residues other than the magnetic particles to the waste-discarding region 39. Please note that the above-mentioned wash process can be optionally performed several times.

Consequently, the extracting module 33 rotates to the position as shown in FIG. 4D, and the pipetting module 32 adds elution buffer in the above-mentioned eppendorf tubes 40 to elute the target genetic material from the magnetic particles. Afterward, the extracting module 33 rotates to be moved close to the first set of receptacles 312 and enforces the magnetic field again, and allows the magnetic particles to be attached on the inner wall of the eppendorf tubes 40. At that time, the pipetting module 32 sucks the eluted target genetic material and moves it to the eppenforf tubes 40 of the second set of receptacles 314.

Consequently, the pipetting module 32 moves suitable volume of target genetic material from the eppendorf tubes 40 of the second set of receptacles 314 to the eppendorf tubes 40 (already contained second reagent) of the third set of receptacles 316. Finally, as shown in FIG. 4F, the moving module 318 moves the platform 31 to allow the third set of receptacles 316 enter the reaction chamber 382 and the door 380 is disposed close to the opening of the reaction chamber 382. Then, the temperature-control module 34 provides pre-set thermal cycles (e.g. 95° C.-54° C.-72° C.) to amplify the target nucleic acid sequence of the target genetic material. As mentioned above, the optical detection module 35 can detect the presence of the target nucleic acid sequence simultaneously or after the amplification, and the result of the detection can be used to qualify and/or quantify the target nucleic acid sequence.

Practically, the automated system for processing genetic material can optionally provide a vacuum environment, a germfree environment, a negative pressure environment or other suitable environment to perform the above-mentioned experiments, so as to prevent the error caused by environment factors. It should be noted that the automated system of the invention can optionally include other suitable modules or members and the design and disposition of said modules or members can optionally be adjusted, but not limited to, the above-mentioned embodiments.

To sum up, the automated system and method for processing genetic material of the invention can extract the genetic material and amplify the nucleic acid sequence with completely automatic processes. Accordingly, the system and method of the invention can simplify the related processes of the prior art, and can reduce the error and bias generated by artificial operation. Additionally, the automated system of the invention can process sets of samples simultaneously, so as to reduce the operation time.

Although the present invention has been illustrated and described with reference to the preferred embodiment thereof, it should be understood that it is in no way limited to the details of such embodiment but is capable of numerous modifications within the scope of the appended claims.

What is claimed is:

1. An automated system for processing genetic material, comprising:
a platform comprising a first set of receptacles, each of which is capable of containing a sample;
a pipetting module comprising at least a pipet, for moving at least a first reagent to the first set of receptacles;
an extracting module, for cooperating with the first reagent to extract a target genetic material from the sample;
a temperature-control module, for providing a plurality of thermal cycles and cooperating with at least a second reagent to amplify a target nucleic acid sequence of the target genetic material;
an optical detection module, for detecting the presence of the target nucleic acid sequence, and generating a detecting signal; and
a processing module, for receiving the detecting signal, and qualifying and/or quantifying the target nucleic acid sequence.

2. The automated system for processing genetic material of claim 1, further comprising:
a housing containing the platform, the pipetting module, the extracting module, the temperature-control module, and the optical detection module.

3. The automated system for processing genetic material of claim 1, further comprising:
a pipet tip container containing a plurality of tips which each has a scale, wherein the pipetting module selects one of the tips to suck or to move the sample, the first reagent, and the second reagent.

4. The automated system for processing genetic material of claim 1, further comprising:
a reagent container comprising a plurality of reagent receptacles for containing the first reagent and/or the second reagent.

5. The automated system for processing genetic material of claim 1, further comprising:
a first moving module, connected to the pipetting module, for horizontally or vertically moving the pipetting module and positioning the pipetting module.

6. The automated system for processing genetic material of claim 1, further comprising:
a second moving module connected to the platform for horizontally moving the platform and positioning the platform.

7. The automated system for processing genetic material of claim 1, further comprising:
a PCR reaction module, disposed adjacent to the temperature-control module and the optical detection module, for sealing the second reagent and the target genetic material, and for receiving the thermal cycles to amplify the target nucleic acid sequence, and the optical detection module being capable of immediately detecting the presence of the target nucleic acid sequence.

8. The automated system for processing genetic material of claim 1, further comprising:
a waste-discarding region, for containing a waste material or a liquid waste.

9. The automated system for processing genetic material of claim 1, further comprising:
a second set of receptacles, for containing the target genetic material; and
a third set of receptacles, for containing the target genetic material and the second reagent.

10. The automated system for processing genetic material of claim 9, wherein the second set of receptacles is formed of a non-magnetic material with low thermal conductivity.

11. The automated system for processing genetic material of claim 9, wherein the third set of receptacles is formed of a non-magnetic material with high thermal conductivity.

12. The automated system for processing genetic material of claim 9, wherein the second reagent is added in the third set of receptacles in advance.

13. The automated system for processing genetic material of claim 1, wherein the first set of receptacles is formed of a non-magnetic material with high thermal conductivity.

14. The automated system for processing genetic material of claim 1, wherein the extracting module is capable of generating a magnetic field.

15. The automated system for processing genetic material of claim 1, wherein the extracting module is rotatably mounted adjacent to the platform, and the extracting module being optionally disposed close to the first set of receptacles.

16. The automated system for processing genetic material of claim 1, wherein the first reagent is selected from a group consisting of PK solution, cell lysis buffer, wash buffer, elution buffer and magnetic particle solution.

17. An automated method for processing genetic material, comprising the following steps of:
(a) mixing a sample with at least a first reagent to extract a target genetic material from the sample;
(b) mixing the target genetic material with at least a second reagent;
(c) providing a plurality of thermal cycles to amplify a target nucleic acid sequence of the target genetic material;
(d) detecting the presence of the target nucleic acid sequence and generating a detecting signal; and
(e) receiving the detecting signal and qualifying and/or quantifying the target nucleic acid sequence.

18. The automated method for processing genetic material of claim 17, wherein step (a) further comprises the following step:
(a1) mixing the sample and the first reagent to form a mixed solution and selectively enforcing a magnetic field to the mixed solution to isolate the target genetic material from the sample.

19. The automated method for processing genetic material of claim 17, wherein the sample and the first reagent in step (a) and the target genetic material and the second reagent in step (b) are mixed via a pipetting module.

20. The automated method for processing genetic material of claim 17, wherein step (c) is performed in a PCR reaction module.

21. The automated method for processing genetic material of claim 17, wherein step (d) is performed by an optical module.

22. The automated method for processing genetic material of claim 17, wherein step (e) is performed by a processing module.

23. The automated method for processing genetic material of claim 17, wherein step (c) to step (e) are performed simultaneously or sequentially.

24. The automated method for processing genetic material of claim 17, wherein the first reagent is selected from a group consisting of PK solution, cell lysis buffer, wash buffer, elution buffer and magnetic particle solution.
